(19) United States
(12) Patent Application Publication
(10) Pub. No.: US 2014/0275169 A1
(43) Pub. Date: Sep. 18, 2014

(54) Combination Therapies for Enhancing Protein Degradation

(71) Applicants: Daniel J. Finley, Jamaica Plain, MA (US); Jessica Robert, Brookline, MA (US); Monica Boselli, Somerville, MA (US); Jason E. Gestwicki, Ann Arbor, MI (US); Xiaokai Li, Ann Arbor, MI (US)

(72) Inventors: Daniel J. Finley, Jamaica Plain, MA (US); Jessica Robert, Brookline, MA (US); Monica Boselli, Somerville, MA (US); Jason E. Gestwicki, Ann Arbor, MI (US); Xiaokai Li, Ann Arbor, MI (US)

(73) Assignees: The Regents of the University of Michigan, Ann Arbor, MI (US); President and Fellows of Harvard College, Cambridge, MA (US)

(21) Appl. No.: 14/208,034

(22) Filed: Mar. 13, 2014

Related U.S. Application Data
(60) Provisional application No. 61/779,314, filed on Mar. 13, 2013.

(51) Int. Cl. A61K 31/454; C07D 417/14; C07D 207/333; A61K 31/4439

(52) U.S. Cl. CPC A61K 31/454; A61K 31/4439; C07D 417/14; C07D 207/333
USPC 514/326; 435/375

(57) ABSTRACT

Disclosed herein are combination therapies for enhancing protein degradation. One component is an inhibitor of USP14, while the second component is an inhibitor of Hsp70. In certain embodiments, the invention relates to methods of treating or preventing a tauopathy, such as Alzheimer's disease.

### RELATED APPLICATIONS

This application claims the benefit of priority to U.S. Provisional Patent Application Ser. No. 61/779,314, filed Mar. 13, 2013; the contents of which are hereby incorporated by reference.

### GOVERNMENT SUPPORT

This invention was made with government support under Grant Nos. R01NS059690 and R01GM095526 awarded by the National Institutes of Health. The government has certain rights in this invention.

### BACKGROUND

### The Proteasome and Usp14

The proteasome is a large protein complex that contains 33 distinct subunits. Proteasome complexes function as proteases in part to degrade unneeded or misfolded proteins. Proteasomes regulate many aspects of cell physiology, and proteasome dysfunction has been implicated in a variety of diseases.

Most, but not all, proteasome substrates are targeted for degradation via the covalent attachment of multimeric chains of a small, highly-conserved protein called ubiquitin. Because longer ubiquitin chains interact more strongly with the proteasome than shorter chains, processes that alter ubiquitin chain length frequently also affect substrate degradation rates. The length of ubiquitin chains attached to substrates tagged for proteasome degradation can be modulated by certain proteasome-associated deubiquitinating enzymes and ubiquitin ligases.

Mammalian proteasomes contain three major deubiquitinating enzymes: Rpn11, Uch37, and Usp14. Usp14 is located in the cytoplasm and cleaves the ubiquitin moiety from ubiquitin-fused precursors and ubiquitinylated proteins.

### Heat Shock Proteins

Heat shock proteins are a class of functionally related proteins involved in the folding and unfolding of other proteins. In addition to protecting cells from thermal or oxidative stress, heat shock proteins stabilize proteins in their partially synthesized or partially unfolded states. Heat shock proteins are classified according to their molecular weights (e.g., 70 kilodalton heat shock proteins (“Hsp70”)). Hsp70 is considered a major “triage” chaperone because it links misfolded proteins to the proteasome for degradation. For example, Hsp70 binds directly to the ubiquitin E3 ligase C-terminus of Hsp70 interacting protein (CHIP) and accelerates ubiquitinylation of Hsp70 clients.

Studies suggest that allosteric inhibition of Hsp70 by small molecule modulators leads to enhanced degradation of Hsp70 clients through the proteasome, thereby improving turnover of these proteins in mammalian cells.

### Tau Tangles

Tau proteins are proteins that stabilize microtubules. They are abundant in neurons of the central nervous system (CNS) and are less common elsewhere. Hyperphosphorylation of the tau protein can result in the assembly of tangles of paired helical filaments and straight filaments, which are involved in the pathogenesis of Alzheimer's disease and other tauopathies.

There exists a need for compounds and methods that stimulate degradation of unwanted, misfolded, or potentially toxic proteins, such as pathogenic forms of tau; these therapies can be used to treat or prevent tauopathies, such as Alzheimer's disease, as well as other diseases of protein misfolding.

### SUMMARY

In certain embodiments, the invention relates to a method of treating or preventing a tauopathy in a subject comprising co-administering to the subject

an effective amount of a first compound, or a pharmaceutically acceptable salt, solvate, hydrate, prodrug, chemically-protected form, enantiomer or stereoisomer thereof, and

an effective amount of a second compound, or a pharmaceutically acceptable salt, solvate, hydrate, prodrug, chemically-protected form, enantiomer or stereoisomer thereof,

wherein the first compound inhibits Usp14 and the second compound inhibits Hsp70.

In certain embodiments, the invention relates to any one of the aforementioned methods, wherein the tauopathy is selected from the group consisting of Alzheimer's Disease (AD), progressive supranuclear palsy, post-encephalitic parkinsonism (PEP), parkinsonism-dementia complex of Guam (PDC Guam), Guadeloupean Parkinsonism, Down's Syndrome, Familial British Dementia, Familial Danish Dementia, Myotonic Dystrophy, Niemann-Pick type C, dementia pugilistica (chronic traumatic encephalopathy), frontotemporal dementia, Parkinson's Disease, Lytico-Bodig disease, tangle-predominant dementia, ganglioglioma, gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), and frontotemporal lobar degeneration.

In certain embodiments, the invention relates to a method of treating or preventing a trinucleotide repeat disorder in a subject comprising co-administering to the subject

an effective amount of a first compound, or a pharmaceutically acceptable salt, solvate, hydrate, prodrug, chemically-protected form, enantiomer or stereoisomer thereof, and

an effective amount of a second compound, or a pharmaceutically acceptable salt, solvate, hydrate, prodrug, chemically-protected form, enantiomer or stereoisomer thereof,

wherein the first compound inhibits Usp14; and the second compound inhibits Hsp70.

In certain embodiments, the invention relates to any one of the aforementioned methods, wherein the trinucleotide repeat disorder is a polyglutamine disease selected from the group consisting of dentatorubropallidoluysian atrophy, Huntington's disease, spinobulbar muscular atrophy, and spinocerebellar ataxia.

In certain embodiments, the invention relates to a method of enhancing degradation of a protein in a cell, comprising contacting the cell with

an effective amount of a first compound, or a pharmaceutically acceptable salt, solvate, hydrate, prodrug, chemically-protected form, enantiomer or stereoisomer thereof, and

an effective amount of a second compound, or a pharmaceutically acceptable salt, solvate, hydrate, prodrug, chemically-protected form, enantiomer or stereoisomer thereof,

wherein the first compound inhibits Usp14 and the second compound inhibits Hsp70.

In certain embodiments, the invention relates to any one of the aforementioned methods, wherein the protein is selected from the group consisting of Tau and polyglutamine.

### BRIEF DESCRIPTION OF THE DRAWINGS

FIG. 1 depicts the synergistic effect of IU1-47 and MKT-077 in stimulating the disappearance (via proteasome-mediated degradation) of tau-A152T, a natural tau mutant in which alanine-152 is substituted with threonine. This experiment was carried out in human-derived neuroblastoma cells (SK—N—SH cells) infected with lentivirus expressing a mutant form of human tau. Tau was detected with the tau-5 antibody. SK—N—SH cells were incubated for one day following the addition of both compounds to the medium. The cells were then lysed, and proteins from the whole-cell extract were resolved by SDS-PAGE followed by immunoblot analysis. Actin was probed as a control showing equivalent amounts of protein loaded from lane to lane; actin is not understood to be affected by IU1-47 or MKT-077.

FIG. 2 depicts a rationale of combination treatment for stimulating tau degradation. Tau (large square), ubiquitin (small tube). The combination treatment is intended to drive the accumulation of ubiquitinated tau, the substrate of the proteasome. According to the rationale, if one only inhibits the DUB pathway, then degradation is limited by slow ubiquitination, and if one only accelerates tau ubiquitination, then degradation will be limited by deubiquitination. MKT-077 and JG48 promote tau ubiquitination (arrows).

FIG. 3 depicts the structures of MKT-077 and JG48.

FIG. 4 depicts the structures of IU1 and IU1-47.

FIG. 5 A and B depict the synergistic effect of IU1-47 and JG48 in stimulating degradation of endogenous total tau. These independently duplicated experiments were carried out in primary cortical rat neurons for 24 hours. Tau was detected after SDS-PAGE immunoblot analysis, using the tau-5 antibody.

The combined effect of Usp14 inhibitors and Hsp70 inhibitors on tau levels is shown to be synergistic in this experiment in that this effect (lane 4) is greater than the sum of the individual effects of these compounds (lanes 2 and 3). To cite one example, in FIG. 5A the individual effects are approximately 8% and 2% for Usp14 and Hsp70 inhibitors, respectively, while the combined effect is approximately 71%, far greater than the expected additive effect of approximately 10%.

FIG. 6 depicts the synergistic effect of IU1-47 and JG48 in stimulating degradation of endogenous phosphorylated tau (A) and endogenous non-phosphorylated tau (B). These experiments were carried out in primary cortical rat neurons for 24 hours. Tau was detected after SDS-PAGE immunoblot analysis, using the PHF-1 (A) and tau1 (B) antibodies.

FIG. 7 depicts the synergistic effect of IU1-47 and JG48 in stimulating degradation of endogenous total tau. This experiment was carried out in primary cortical rat neurons for 48 hours. Tau was detected after SDS-PAGE immunoblot analysis, using the tau-5 antibody.

FIG. 8 depicts the synergistic effect of IU1-47 and JG48 in stimulating degradation of endogenous total tau. This experiment was carried out in primary cortical rat neurons for 24 hours. Tau was detected after SDS-PAGE immunoblot analysis, using the tau-5 antibody. This experiment, performed with lower doses of both inhibitors, shows that synergism is robust, as it is exhibited over a range of doses.

FIG. 9 depicts the combined effect of IU1-47 and JG48 in neuron viability. These experiments were carried out in duplicate in primary cortical rat neurons for 24 hours.

FIG. 10 depicts exemplary second compounds of the invention.

### DETAILED DESCRIPTION

### Overview

In certain embodiments, the invention relates to the effect of small molecules on protein degradation. The ubiquitin-proteasome system (UPS) is a key player in maintaining cellular protein homeostasis and is associated with various human diseases, including neurodegenerative disorders, cancer, and infectious diseases.

IU1, or 1-(1-(4-fluorophenyl)-2,5-dimethyl-1H-pyrrol-3-yl)-2-(pyrrolidin-1-yl)ethanone, is an inhibitor of Usp14, a deubiquitinating enzyme that resides on the proteasome. IU1 has been shown to enhance proteasome activity. IU1-47 is a closely related and more potent variant of IU1. Both IU1 and IU1-47 promote the degradation of the protein tau.

In certain embodiments, under conditions in which the IU1-47 effect on tau degradation is modest, tau degradation is accentuated by combined treatment with an inhibitor of Hsp70 (either MKT-077 (FIG. 1) or JG48 (FIGS. 5-8)). For the sake of brevity, we may refer at times to IU1-47 and JG48 to exemplify properties of Usp14 inhibitors and Hsp70 allosteric inhibitors, respectively. Insofar as analogous compounds of greater or less potency exist, those compounds are also expected to act synergistically in the assays described herein and are included in this invention. In certain embodiments, the two compounds act synergistically to stimulate tau degradation. Although tau is expressed from a lentivirus construct in the studies presented in FIG. 1, the ability of both IU1-47 and MKT-077 to enhance tau degradation is not peculiar to overexpressed forms of tau. For example, see FIGS. 5-8, where JG48 is used in place of MKT-077.

While not wishing to be bound by any particular theory, MKT-077 is thought to stimulate tau ubiquitination, a process that is antagonized by deubiquitinating enzymes. The stimulation of tau ubiquitination and the inhibition of deubiquitination by IU1-47 should mutually reinforce each other (FIG. 2). In certain embodiments, the combined use of these compounds—Usp14 inhibitors and Hsp70 inhibitors—could be efficacious in the treatment of tauopathies. In addition, in certain embodiments, the method may be applicable to toxic proteins other than tau.

As can be inferred from FIG. 9, the synergism of combining Usp14 inhibiton with Hsp70 inhibition can be achieved without an overt synergistic effect on the toxicity of these compounds. Thus, combination treatment may allow both compounds to be effective at a lower dose, and for shorter periods of time, thus potentially minimizing detrimental effects of compound treatment.

### DEFINITIONS

In order for the invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The definition of each expression, e.g., alkyl, m, n, and the like, when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., a compound which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein below. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The term “lower” when appended to any of the groups listed below indicates that the group contains less than seven carbons (i.e. six carbons or less). For example “lower alkyl” refers to an alkyl group containing 1-6 carbons, and “lower alkenyl” refers to an alkyenyl group containing 2-6 carbons.

The term “saturated,” as used herein, pertains to compounds and/or groups which do not have any carbon-carbon double bonds or carbon-carbon triple bonds.

The term “unsaturated,” as used herein, pertains to compounds and/or groups which have at least one carbon-carbon double bond or carbon-carbon triple bond.

The term “aliphatic,” as used herein, pertains to compounds and/or groups which are linear or branched, but not cyclic (also known as “acyclic” or “open-chain” groups).

The term “cyclic,” as used herein, pertains to compounds and/or groups which have one ring, or two or more rings (e.g., spiro, fused, bridged).

The term “aromatic” refers to a planar or polycyclic structure characterized by a cyclically conjugated molecular moiety containing 4n+2 electrons, wherein n is the absolute value of an integer. Aromatic molecules containing fused, or joined, rings also are referred to as bicylic aromatic rings. For example, bicyclic aromatic rings containing heteroatoms in a hydrocarbon ring structure are referred to as bicyclic heteroaryl rings.

The term “hydrocarbon” as used herein refers to an organic compound consisting entirely of hydrogen and carbon.

For purposes of this invention, the chemical elements are identified in accordance with the Periodic Table of the Elements, CAS version, Handbook of Chemistry and Physics, 67th Ed., 1986-87, inside cover.

The term “heteroatom” as used herein is art-recognized and refers to an atom of any element other than carbon or hydrogen. Illustrative heteroatoms include boron, nitrogen, oxygen, phosphorus, sulfur and selenium.

The term “alkyl” means an aliphatic or cyclic hydrocarbon radical containing from 1 to 12 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 2-methylcyclopentyl, and 1-cyclohexylethyl.

The term “substituted alkyl” means an aliphatic or cyclic hydrocarbon radical containing from 1 to 12 carbon atoms, substituted with 1, 2, 3, 4, or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, halo alkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfiny, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, halo alkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, halo alkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl and silyloxy.

The term “alkylene” is art-recognized, and as used herein pertains to a bidentate moiety obtained by removing two hydrogen atoms of an alkyl group, as defined above.

The term “alkenyl” as used herein means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkynyl” as used herein means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “carbocyclyl” as used herein means monocyclic or multicyclic (e.g., bicyclic, tricyclic, etc.) hydrocarbons containing from 3 to 12 carbon atoms that is completely saturated or has one or more unsaturated bonds, and for the avoidance of doubt, the degree of unsaturation does not result in an aromatic ring system (e.g. phenyl). Examples of carbocyclyl groups include 1-cyclopropyl, 1-cyclobutyl, 2-cyclopentyl, 1-cyclopentenyl, 3-cyclohexyl, 1-cyclohexenyl and 2-cyclopentenylmethyl.

The term “heterocyclyl”, as used herein include non-aromatic, ring systems, including, but not limited to, monocyclic, bicyclic (e.g. fused and spirocyclic) and tricyclic rings, which can be completely saturated or which can contain one or more units of unsaturation, for the avoidance of doubt, the degree of unsaturation does not result in an aromatic ring system, and have 3 to 12 atoms including at least one heteroatom, such as nitrogen, oxygen, or sulfur. For purposes of exemplification, which should not be construed as limiting the scope of this invention, the following are examples of heterocyclic rings: azepines, azetidinyl, morpholinyl, oxopiperidinyl, oxopyrrolidinyl, piperazinyl, piperidinyl, pyrrolidinyl, quinicludinyl, thiomorpholinyl, tetrahydropyranyl and tetrahydrofuranyl. The heterocyclyl groups of the invention are substituted with 0, 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, halo alkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfiny, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, halo alkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, halo alkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the heterocyclyl group through an alkylene moiety (e.g. methylene).

The term “N-heterocyclyl” as used herein is a subset of heterocyclyl, as defined herein, which have at least one nitrogen atom through which the N-heterocyclyl moiety is bound to the parent moiety. Representative examples include pyrrolidin-1-yl, piperidin-1-yl, piperazin-1-yl, hexahydropyrimidin-1-yl, morpholin-1-yl, 1,3-oxazinan-3-yl and 6-azaspiro[2.5]oct-6-yl. As with the heterocyclyl groups, the N-heterocyclyl groups of the invention are substituted with 0, 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfiny, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, halo alkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, halo alkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the N-heterocyclyl group through an alkylene moiety (e.g. methylene).

The term “aryl,” as used herein means a phenyl group, naphthyl or anthracenyl group. The aryl groups of the invention can be optionally substituted with 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfiny, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, halo alkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, halo alkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the heterocyclyl group through an alkylene moiety (e.g. methylene).

The term “arylene,” is art-recognized, and as used herein pertains to a bidentate moiety obtained by removing two hydrogen atoms of an aryl ring, as defined above.

The term “arylalkyl” or “aralkyl” as used herein means an aryl group, as defined herein, appended to the parent molecular moiety through an alkyl group, as defined herein. Representative examples of aralkyl include, but are not limited to, benzyl, 2-phenylethyl, 3-phenylpropyl, and 2-naphth-2-ylethyl.

The term “biaryl,” as used herein means an aryl-substituted aryl, an aryl-substituted heteroaryl, a heteroaryl-substituted aryl or a heteroaryl-substituted heteroaryl, wherein aryl and heteroaryl are as defined herein. Representative examples include 4-(phenyl)phenyl and 4-(4-fluorophenyl)pyridinyl.

The term “heteroaryl” as used herein include aromatic ring systems, including, but not limited to, monocyclic, bicyclic and tricyclic rings, and have 3 to 12 atoms including at least one heteroatom, such as nitrogen, oxygen, or sulfur. For purposes of exemplification, which should not be construed as limiting the scope of this invention: azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl. The heteroaryl groups of the invention are substituted with 0, 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, sulfhydryl, alkylthio, haloalkylthio, fluoroalkylthio, alkenylthio, alkynylthio, sulfonic acid, alkylsulfonyl, haloalkylsulfonyl, fluoroalkylsulfonyl, alkenylsulfonyl, alkynylsulfonyl, alkoxysulfonyl, halo alkoxysulfonyl, fluoroalkoxysulfonyl, alkenyloxysulfonyl, alkynyloxysulfonyl, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfiny, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, halo alkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, halo alkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the heteroaryl group through an alkylene moiety (e.g. methylene).

The term “heteroarylene,” is art-recognized, and as used herein pertains to a bidentate moiety obtained by removing two hydrogen atoms of a heteroaryl ring, as defined above.

The term “heteroarylalkyl” or “heteroaralkyl” as used herein means a heteroaryl, as defined herein, appended to the parent molecular moiety through an alkyl group, as defined herein. Representative examples of heteroarylalkyl include, but are not limited to, pyridin-3-ylmethyl and 2-(thien-2-yl)ethyl.

The term “halo” or “halogen” means —Cl, —Br, —I or —F.

The term “haloalkyl” means an alkyl group, as defined herein, wherein at least one hydrogen is replaced with a halogen, as defined herein. Representative examples of haloalkyl include, but are not limited to, chloromethyl, 2-fluoroethyl, trifluoromethyl, pentafluoroethyl, and 2-chloro-3-fluoropentyl.

The term “fluoroalkyl” means an alkyl group, as defined herein, wherein all the hydrogens are replaced with fluorines.

The term “hydroxy” as used herein means an —OH group.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy. The terms “alkenyloxy”, “alkynyloxy”, “carbocyclyloxy”, and “heterocyclyloxy” are likewise defined.

The term “haloalkoxy” as used herein means an alkoxy group, as defined herein, wherein at least one hydrogen is replaced with a halogen, as defined herein. Representative examples of haloalkoxy include, but are not limited to, chloromethoxy, 2-fluoroethoxy, trifluoromethoxy, and pentafluoroethoxy. The term “fluoroalkyloxy” is likewise defined.

The term “aryloxy” as used herein means an aryl group, as defined herein, appended to the parent molecular moiety through an oxygen. The term “heteroaryloxy” as used herein means a heteroaryl group, as defined herein, appended to the parent molecular moiety through an oxygen. The terms “heteroaryloxy” is likewise defined.

The term “arylalkoxy” or “arylalkyloxy” as used herein means an arylalkyl group, as defined herein, appended to the parent molecular moiety through an oxygen. The term “heteroarylalkoxy” is likewise defined. Representative examples of aryloxy and heteroarylalkoxy include, but are not limited to, 2-chlorophenylmethoxy, 3-trifluoromethyl-phenylethoxy, and 2,3-dimethylpyridinylmethoxy.

The term “sulfhydryl” or “thio” as used herein means a —SH group.

The term “alkylthio” as used herein means an alkyl group, as defined herein, appended to the parent molecular moiety through a sulfur. Representative examples of alkylthio include, but are not limited, methylthio, ethylthio, tert-butylthio, and hexylthio. The terms “haloalkylthio”, “fluoroalkylthio”, “alkenylthio”, “alkynylthio”, “carbocyclylthio”, and “heterocyclylthio” are likewise defined.

The term “arylthio” as used herein means an aryl group, as defined herein, appended to the parent molecular moiety through an sulfur. The term “heteroarylthio” is likewise defined.

The term “arylalkylthio” or “aralkylthio” as used herein means an arylalkyl group, as defined herein, appended to the parent molecular moiety through an sulfur. The term “heteroarylalkylthio” is likewise defined.

The term “sulfonyl” as used herein refers to —S(═O)₂— group.

The term “sulfonic acid” as used herein refers to —S(═O)₂OH.

The term “alkylsulfonyl” as used herein means an alkyl group, as defined herein, appended to the parent molecular moiety through a sulfonyl group, as defined herein. Representative examples of alkylsulfonyl include, but are not limited to, methylsulfonyl and ethylsulfonyl. The terms “haloalkylsulfonyl”, “fluoroalkylsulfonyl”, “alkenylsulfonyl”, “alkynylsulfonyl”, “carbocyclylsulfonyl”, “heterocyclylsulfonyl”, “arylsulfonyl”, “aralkylsulfonyl”, “heteroarylsulfonyl” and “heteroaralkylsulfonyl” are likewise defined.

The term “alkoxysulfonyl” as used herein means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl. The terms “haloalkoxysulfonyl”, “fluoroalkoxysulfonyl”, “alkenyloxysulfonyl”, “alkynyloxysulfonyl”, “carbocyclyloxysulfonyl”, “heterocyclyloxysulfonyl”, “aryloxysulfonyl”, “aralkyloxysulfonyl”, “heteroaryloxysulfonyl” and “heteroaralkyloxysulfonyl” are likewise defined.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The term “aminosulfonyl” as used herein means an amino group, as defined herein, appended to the parent molecular moiety through a sulfonyl group.

The term “sulfinyl” as used herein refers to —S(═O)— group. Sulfinyl groups are as defined above for sulfonyl groups. The term “sulfinic acid” as used herein refers to —S(═O)OH.

The term “oxy” refers to a —O— group.

The term “carbonyl” as used herein means a —C(═O)— group.

The term “thiocarbonyl” as used herein means a —C(═S)— group.

The term “formyl” as used herein means a —C(═O)H group.

The term “alkylcarbonyl” as used herein means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl. The terms “haloalkylcarbonyl”, “fluoroalkylcarbonyl”, “alkenylcarbonyl”, “alkynylcarbonyl”, “carbocyclylcarbonyl”, “heterocyclylcarbonyl”, “arylcarbonyl”, “aralkylcarbonyl”, “heteroarylcarbonyl”, and “heteroaralkylcarbonyl” are likewise defined.

The term “carboxy” as used herein means a —CO₂H group.

The term “alkoxycarbonyl” as used herein means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl. The terms “haloalkoxycarbonyl”, “fluoroalkoxycarbonyl”, “alkenyloxycarbonyl”, “alkynyloxycarbonyl”, “carbocyclyloxycarbonyl”, “heterocyclyloxycarbonyl”, “aryloxycarbonyl”, “aralkyloxycarbonyl”, “heteroaryloxycarbonyl”, and “heteroaralkyloxycarbonyl” are likewise defined.

The term “alkylcarbonyloxy” as used herein means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. The terms “haloalkylcarbonyloxy”, “fluoroalkylcarbonyloxy”, “alkenylcarbonyloxy”, “alkynylcarbonyloxy”, “carbocyclylcarbonyloxy”, “heterocyclylcarbonyloxy”, “arylcarbonyloxy”, “aralkylcarbonyloxy”, “heteroarylcarbonyloxy”, and “heteroaralkylcarbonyloxy” are likewise defined.

The term “alkylsulfonyloxy” as used herein means an alkylsulfonyl group, as defined herein, appended to the parent molecular moiety through an oxygen atom. The terms “haloalkylsulfonyloxy”, “fluoroalkylsulfonyloxy”, “alkenylsulfonyloxy”, “alkynylsulfonyloxy”, “carbocyclylsulfonyloxy”, “heterocyclylsulfonyloxy”, “arylsulfonyloxy”, “aralkylsulfonyloxy”, “heteroarylsulfonyloxy”, “heteroaralkylsulfonyloxy”, “halo alkoxysulfonyloxy”, “fluoroalkoxysulfonyloxy”, “alkenyloxysulfonyloxy”, “alkynyloxysulfonyloxy”, “carbocyclyloxysulfonyloxy”, “heterocyclyloxysulfonyloxy”, “aryloxysulfonyloxy”, “aralkyloxysulfonyloxy”, “heteroaryloxysulfonyloxy” and “heteroaralkyloxysulfonyloxy”

The term “amino” as used herein refers to —NH₂ and substituted derivatives thereof wherein one or both of the hydrogens are independently replaced with substituents selected from the group consisting of alkyl, haloalkyl, fluoroalkyl, alkenyl, alkynyl, carbocyclyl, heterocyclyl, aryl, aralkyl, heteroaryl, heteroaralkyl, alkylcarbonyl, haloalkylcarbonyl, fluoroalkylcarbonyl, alkenylcarbonyl, alkynylcarbonyl, carbocyclylcarbonyl, heterocyclylcarbonyl, arylcarbonyl, aralkylcarbonyl, heteroarylcarnbonyl, heteroaralkylcarbonyl and the sufonyl and sulfinyl groups defined above; or when both hydrogens together are replaced with an alkylene group (to form a ring which contains the nitrogen). Representative examples include, but are not limited to methylamino, acetylamino, and dimethylamino.

The term “amido” as used herein means an amino group, as defined herein, appended to the parent molecular moiety through a carbonyl.

The term “cyano” as used herein means a —C≡N group.

The term “nitro” as used herein means a —NO₂ group.

The term “azido” as used herein means a —N₃ group.

The term “phosphinyl” as used herein includes —PH₃ and substituted derivatives thereof wherein one, two or three of the hydrogens are independently replaced with substituents selected from the group consisting of alkyl, haloalkyl, fluoroalkyl, alkenyl, alkynyl, carbocyclyl, heterocyclyl, aryl, aralkyl, heteroaryl, heteroaralkyl, alkoxy, haloalkoxy, fluoroalkyloxy, alkenyloxy, alkynyloxy, carbocyclyloxy, heterocyclyloxy, aryloxy, aralkyloxy, heteroaryloxy, heteroaralkyloxy, and amino.

The term “phosphoryl” as used herein refers to —PO(═O)OH₂ and substituted derivatives thereof wherein one or both of the hydroxyls are independently replaced with substituents selected from the group consisting of alkyl, haloalkyl, fluoroalkyl, alkenyl, alkynyl, carbocyclyl, heterocyclyl, aryl, aralkyl, heteroaryl, heteroaralkyl, alkoxy, haloalkoxy, fluoroalkyloxy, alkenyloxy, alkynyloxy, carbocyclyloxy, heterocyclyloxy, aryloxy, aralkyloxy, heteroaryloxy, heteroaralkyloxy, and amino.

The term “silyl” as used herein includes H₃Si— and substituted derivatives thereof wherein one, two or three of the hydrogens are independently replaced with substituents selected from alkyl, haloalkyl, fluoroalkyl, alkenyl, alkynyl, carbocyclyl, heterocyclyl, aryl, aralkyl, heteroaryl, and heteroaralkyl. Representative examples include trimethylsilyl (TMS), tert-butyldiphenylsilyl (TBDPS), tert-butyldimethylsilyl (TBS/TBDMS), triisopropylsilyl (TIPS), and [2-(trimethylsilyl)ethoxy]methyl (SEM).

The term “silyloxy” as used herein means a silyl group, as defined herein, is appended to the parent molecule through an oxygen atom.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

As used herein, the term “administering” means providing a pharmaceutical agent or composition to a subject, and includes, but is not limited to, administering by a medical professional and self-administering.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As used herein, the phrase “pharmaceutically-acceptable salts” refers to the relatively non-toxic, inorganic and organic salts of compounds.

As used herein, the term “subject” means a human or non-human animal selected for treatment or therapy.

As used herein, the phrase “subject suspected of having” means a subject exhibiting one or more clinical indicators of a disease or condition.

As used herein, the phrase “subject in need thereof” means a subject identified as in need of a therapy or treatment of the invention.

As used herein, the phrase “therapeutic effect” refers to a local or systemic effect in animals, particularly mammals, and more particularly humans, caused by an agent. The phrases “therapeutically-effective amount” and “effective amount” mean the amount of an agent that produces some therapeutically useful effect on the symptoms of the tauopathy. A therapeutically effective amount includes an amount of an agent that produces some desired local or systemic effect at a reasonable benefit/risk ratio applicable to any treatment. For example, certain agents used in the methods of the invention may be administered in a sufficient amount to produce a reasonable benefit/risk ratio applicable to such treatment.

As used herein, the term “treating” a disease in a subject or “treating” a subject having or suspected of having a disease refers to subjecting the subject to a pharmaceutical treatment, e.g., the administration of an agent, such that at least one symptom of the disease is decreased or prevented from worsening.

### Therapeutic Methods of the Invention

In certain embodiments, the invention relates to a method of treating or preventing a tauopathy in a subject comprising co-administering to the subject (e.g., a subject in need thereof), an effective amount of a first compound, or a pharmaceutically acceptable salt, solvate, hydrate, prodrug, chemically-protected form, enantiomer or stereoisomer thereof, and an effective amount of a second compound, or a pharmaceutically acceptable salt, solvate, hydrate, prodrug, chemically-protected form, enantiomer or stereoisomer thereof, wherein the first compound inhibits Usp14; and the second compound inhibits Hsp70.

In certain embodiments, the invention relates to any one of the aforementioned methods, wherein the tauopathy is selected from the group consisting of Alzheimer's Disease (AD), progressive supranuclear palsy, post-encephalitic parkinsonism (PEP), parkinsonism-dementia complex of Guam (PDC Guam), Guadeloupean Parkinsonism, Down's Syndrome, Familial British Dementia, Familial Danish Dementia, Myotonic Dystrophy, Niemann-Pick type C, dementia pugilistica (chronic traumatic encephalopathy), frontotemporal dementia, Parkinson's Disease, Lytico-Bodig disease, tangle-predominant dementia, ganglioglioma, gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), and frontotemporal lobar degeneration.

In certain embodiments, the invention relates to any one of the aforementioned methods, wherein the tauopathy is frontotemporal dementia with parkinsonism-17 (FTDP-17).

In certain embodiments, the invention relates to a method of treating or preventing a trinucleotide repeat disorder in a subject comprising co-administering to the subject (e.g., a subject in need thereof), an effective amount of a first compound, or a pharmaceutically acceptable salt, solvate, hydrate, prodrug, chemically-protected form, enantiomer or stereoisomer thereof, and an effective amount of a second compound, or a pharmaceutically acceptable salt, solvate, hydrate, prodrug, chemically-protected form, enantiomer or stereoisomer thereof, wherein the first compound inhibits Usp14; and the second compound inhibits Hsp70.

In certain embodiments, the invention relates to any one of the aforementioned methods, wherein the trinucleotide repeat disorder is polyglutamine disease.

In certain embodiments, the invention relates to any one of the aforementioned methods, wherein the polyglutamine disease is selected from the group consisting of dentatorubropallidoluysian atrophy, Huntington's disease, spinobulbar muscular atrophy, and spinocerebellar ataxia (e.g., Type 1, Type 2, Type 3, Type 6, Type 7, or Type 17).

In certain embodiments, the invention relates to any one of the aforementioned methods, wherein the trinucleotide repeat disorder is selected from the group consisting of Fragile X syndrome, Fragile X-associated tremor/ataxia syndrome, Fragile XE mental retardation, Friedreich's ataxia, myotonic dystrophy, spinocerebellar ataxia Type 8, and spinocerebellar ataxia Type 12.

In certain embodiments, the invention relates to a method of enhancing degradation of a protein in a cell, comprising contacting the cell (e.g., a cell in need thereof), with an effective amount of a first compound, or a pharmaceutically acceptable salt, solvate, hydrate, prodrug, chemically-protected form, enantiomer or stereoisomer thereof, and an effective amount of a second compound, or a pharmaceutically acceptable salt, solvate, hydrate, prodrug, chemically-protected form, enantiomer or stereoisomer thereof, wherein the first compound inhibits Usp14 and the second compound inhibits Hsp70.

In certain embodiments, the invention relates to any one of the aforementioned methods, wherein the protein is a misfolded protein. In certain embodiments, the invention relates to any one of the aforementioned methods, wherein the protein is a chronically misfolded protein.

In certain embodiments, the invention relates to any one of the aforementioned methods, wherein the protein is selected from the group consisting of Tau and polyglutamine (polyQ).

In certain embodiment, the invention relates to any one of the aforementioned methods, wherein the first compound is represented by Formula I

[chemical structure]

wherein, independently for each occurrence,

A is aryl, heteroaryl, carbocyclyl, heterocyclyl, or biaryl;

R¹ is hydrogen, alkyl, haloalkyl, fluoroalkyl, lower alkoxy, halo or trifluoromethyl;

G is —N═ or —C(R²)═;

Z is ═C(R⁸)—, ═C(R²)— or ═N—;

R² is hydrogen, alkyl, haloalkyl, fluoroalkyl, lower alkoxy, halo or trifluoromethyl; or, when G is —C(R²)═ and Z is ═C(R²)—, the two R² taken together are

[chemical structure]

X is

[chemical structure]

or heteroaryl;

Y is —CH₂NR³R⁴, —CH₂(N-heterocyclyl), —CH₂NH(CH₂)ₙNH(alkyl), —CH₂NH(CH₂)ₙN(alkyl)₂, —CH₂NH(CH₂)ₙ(N-heterocyclyl), —CH₂N(alkyl)(CH₂)ₙNH(alkyl), —CH₂N(alkyl)(CH₂)ₙN(alkyl)₂, —CH₂N(alkyl)(CH₂)ₙ(N-heterocyclyl), —CH₂NH(CH₂)ₙO(alkyl), —CH₂N(alkyl)(CH₂)ₙO(alkyl), —NR³R⁴, —NR⁵NR⁶R⁷, —NR⁵(N-heterocyclyl), or —N-heterocyclyl;

n is 1, 2, 3 or 4;

R³ is hydrogen, alkyl, substituted alkyl, alkoxyalkyl, haloalkyl, fluoroalkyl, aryl, aralkyl, heteroaryl, or heteroaralkyl;

R⁴ is hydrogen, alkyl, substituted alkyl, alkoxyalkyl, haloalkyl, fluoroalkyl, aryl, aralkyl, heteroaryl, or heteroaralkyl;

R⁵ is hydrogen, alkyl, substituted alkyl, alkoxyalkyl, haloalkyl, fluoroalkyl, aryl, aralkyl, heteroaryl, or heteroaralkyl;

R⁶ is hydrogen, alkyl, substituted alkyl, alkoxyalkyl, haloalkyl, fluoroalkyl, aryl, aralkyl, heteroaryl, or heteroaralkyl;

R⁷ is hydrogen, alkyl, substituted alkyl, alkoxyalkyl, haloalkyl, fluoroalkyl, aryl, aralkyl, heteroaryl, or heteroaralkyl;

R⁸ is hydrogen, alkyl, substituted alkyl, alkoxyalkyl, haloalkyl, fluoroalkyl, aryl, aralkyl, heteroaryl, or heteroaralkyl;

R⁹ is alkyl; or two R⁹ taken together with the nitrogen to which they are bound are an N-heterocyclyl group; and

R¹⁰ is hydrogen, alkyl, haloalkyl, fluoroalkyl, alkoxy, alkoxyalkyl, halo, trifluoromethyl, sulfoxymethyl, sulfonamido, amino, amido, N-heterocyclyl, aminoalkyl, amidoalkyl, or N-hetrocyclylalkyl.

In certain embodiments, the invention relates to any of the aforementioned methods, wherein G is —N═.

In certain embodiments, the invention relates to any of the aforementioned methods, wherein G is —C(R²)═.

In certain embodiments, the invention relates to any of the aforementioned methods, wherein the first compound is represented by Formula II:

[chemical structure]

In certain embodiments, the invention relates to any of the aforementioned methods, wherein the first compound is

[chemical structure]

In certain embodiments, the invention relates to any of the aforementioned methods, wherein the first compound is described in PCT published patent application number WO11/094545, which is incorporated herein by reference in its entirety.

In certain embodiments, the invention relates to any of the aforementioned methods, wherein the second compound is represented by Formula III

[chemical structure]

wherein

A′ is a substituted heteroaromatic moiety;

B′ is a substituted heteroaromatic moiety; and

C′ is a substituted heteroaromatic moiety.

In certain embodiments, the invention relates to any of the aforementioned methods, wherein the compound of Formula III is a salt.

In certain embodiments, the invention relates to any of the aforementioned methods, wherein A′ is a substituted 9-membered heteroaromatic ring. In certain embodiments, the invention relates to any of the aforementioned methods, wherein A′ is a benzothiazole moiety. In certain embodiments, the invention relates to any of the aforementioned methods, wherein A′ is substituted or unsubstituted

[chemical structure]

In certain embodiments, the invention relates to any of the aforementioned methods, wherein A′ is

[chemical structure]

In certain embodiments, the invention relates to any of the aforementioned methods, wherein B′ is a substituted 5-membered heteroaromatic ring. In certain embodiments, the invention relates to any of the aforementioned methods, wherein B′ is a thiazolidinone moiety. In certain embodiments, the invention relates to any of the aforementioned methods, wherein B′ is

[chemical structure]

In certain embodiments, the invention relates to any of the aforementioned methods, wherein C′ is a substituted 6-membered heteroaromatic ring or a substituted 5-membered heteroaromatic ring. In certain embodiments, the invention relates to any of the aforementioned methods, wherein C′ is a pyridine moiety. In certain embodiments, the invention relates to any of the aforementioned methods, wherein C′ is a pyridinium moiety. In certain embodiments, the invention relates to any of the aforementioned methods, wherein C′ is

[chemical structure]

In certain embodiments, the invention relates to any of the aforementioned methods, wherein [symbol] represents a double bond.

In certain embodiments, the invention relates to any one of the aforementioned methods, wherein the second compound is selected from the group consisting of MKT-077, JG48, 2-phenylethyenesulfonamide (PES or pifithrin-μ), 5′-O-[(4-cyanophenyl)methyl]-8-[[(3,4-dichlorophenyl)methyl]amino]-adenosine (VER 155008), methylene blue, azure C, and myricetin.

In certain embodiments, the invention relates to any one of the aforementioned methods, wherein the second compound is selected from the group consisting of

[chemical structure]

[chemical structure]

[chemical structure]

In certain embodiments, the invention relates to any one of the aforementioned methods, wherein the second compound is

[chemical structure]

In certain embodiments, the invention relates to any one of the aforementioned methods, wherein the second compound is

[chemical structure]

The methods of the invention are useful for treating a subject in need thereof. A subject in need thereof is a subject having or at risk of having a tauopathy. In its broadest sense, the terms “treatment” or “to treat” refer to both therapeutic and prophylactic treatments. If the subject in need of treatment is experiencing a condition (i.e., has or is having a particular condition), then “treating the condition” refers to ameliorating, reducing or eliminating one or more symptoms arising from the condition. If the subject in need of treatment is one who is at risk of having a condition, then treating the subject refers to reducing the risk of the subject having the condition or, in other words, decreasing the likelihood that the subject will develop a tauopathy, as well as to a treatment after the subject has developed a tauopathy, e.g., reduce or eliminate it altogether or prevent it from becoming worse.

Thus the invention encompasses the use of the compounds described herein alone or in combination with other therapeutics for the treatment of a subject having or at risk of having a tauopathy.

As used herein, a subject includes humans and non-human animals such as non-human primates, dogs, cats, sheep, goats, cows, pigs, horses and rodents.

In some embodiments, the desired dose of the active agents will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of the active agents may be determined by reference to the plasma concentrations of the agent. For example, the maximum plasma concentration (Cmax) and the area under the plasma concentration-time curve from time 0 to infinity (AUC (0-4)) may be used. Dosages for the invention include those that produce the above values for Cmax and AUC (0-4) and other dosages resulting in larger or smaller values for those parameters.

Actual dosage levels of the active agents may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular agents employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical compositions required. For example, the physician or veterinarian could prescribe and/or administer doses of the agents of the invention employed in the pharmaceutical composition at levels lower than that required in order to achieve the desired therapeutic effect and gradually increase the dosage until the desired effect is achieved.

In general, a suitable daily dose of an agent of the invention will be that amount of the agent (e.g., the compound) that is the lowest dose effective to produce a therapeutic effect. Such an effective dose will generally depend upon the factors described above.

If desired, the effective daily dose of the agent may be administered as two, three, four, five, six or more sub-doses administered separately at appropriate intervals throughout the day, optionally, in unit dosage forms.

The precise time of administration and amount of any particular agent that will yield the most effective treatment in a given patient will depend upon the activity, pharmacokinetics, and bioavailability of a particular agent, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the subject may be monitored by measuring one or more of the relevant indices at predetermined times during a 24-hour period. All aspects of the treatment, including supplements, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments, for example, to the amount(s) of agent administered and to the time of administration may be made based on these reevaluations.

Treatment may be initiated with smaller dosages that are less than the optimum dose of the compounds. Thereafter, the dosage may be increased by small increments until the optimum therapeutic effect is attained.

In certain embodiments, the invention relates to any of the aforementioned methods, wherein A is aryl or heteroaryl.

In certain embodiments, the invention relates to any of the aforementioned methods, wherein A is phenyl, pyridin-2-yl, pyridin-3-yl or pyrimidin-2-yl, optionally substituted with 1, 2, 3, 4 or 5 substituents independently selected from the group consisting of alkyl, alkenyl, alkynyl, halo, haloalkyl, fluoroalkyl, hydroxy, alkoxy, alkenyloxy, alkynyloxy, carbocyclyloxy, heterocyclyloxy, haloalkoxy, fluoroalkyloxy, formyl, alkylcarbonyl, haloalkylcarbonyl, fluoroalkylcarbonyl, alkenylcarbonyl, alkynylcarbonyl, carboxy, alkoxycarbonyl, halo alkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, sulfoxymethyl, sulfonamido, amino, amido, azido, aminosulfonyl, aminosulfinyl, cyano, nitro, phosphinyl, phosphoryl, silyl, silyloxy, and any of said substituents bound to the phenyl, pyridin-2yl, pyridin-3-yl or pyrimidin-2-yl through a methylene or ethylene moiety.

In certain embodiments, the invention relates to any of the aforementioned methods, wherein A is phenyl, optionally substituted with 1, 2, 3, 4 or 5 substituents independently selected from the group consisting of alkyl, halo, haloalkyl, fluoroalkyl, hydroxy, alkoxy, haloalkoxy, fluoroalkyloxy, amino, azido, cyano, and nitro.

In certain embodiments, the invention relates to any of the aforementioned methods, wherein A is

[chemical structure]

In certain embodiments, the invention relates to any of the aforementioned methods, wherein A is phenyl substituted in the two position (ortho substituted) with a substituent selected from the group consisting of alkyl, halo, haloalkyl, fluoroalkyl, hydroxy, alkoxy, haloalkoxy, fluoroalkyloxy, amino, azido, cyano, and nitro.

In certain embodiments, the invention relates to any of the aforementioned methods, wherein A is

[chemical structure]

In certain embodiments, the invention relates to any of the aforementioned methods, wherein A is phenyl substituted in the three position (meta substituted) with a substituent selected from the group consisting of alkyl, halo, haloalkyl, fluoroalkyl, hydroxy, alkoxy, haloalkoxy, fluoroalkyloxy, amino, azido cyano, and nitro.

In certain embodiments, the invention relates to any of the aforementioned methods, wherein A is

[chemical structure]

In certain embodiments, the invention relates to any of the aforementioned methods, wherein A is phenyl substituted in the four position (para substituted) with a substituent selected from the group consisting of alkyl, halo, haloalkyl, fluoroalkyl, hydroxy, alkoxy, haloalkoxy, fluoroalkyloxy, amino, azido, cyano, and nitro.

In certain embodiments, the invention relates to any of the aforementioned methods, wherein A is

[chemical structure]

In certain embodiments, the invention relates to any of the aforementioned methods, wherein A is phenyl substituted in the two and four positions with substituents independently selected from the group consisting of alkyl, halo, haloalkyl, fluoroalkyl, hydroxy, alkoxy, haloalkoxy, fluoroalkyloxy, amino, azido, cyano, and nitro.

In certain embodiments, the invention relates to any of the aforementioned methods, wherein A is

[chemical structure]

In certain embodiments, the invention relates to any of the aforementioned methods, wherein A is pyridin-2-yl, optionally substituted in the four position with a substituent selected from the group consisting of alkyl, halo, haloalkyl, fluoroalkyl, hydroxy, alkoxy, haloalkoxy, fluoroalkyloxy, amino, azido, cyano, and nitro.

In certain embodiments, the invention relates to any of the aforementioned methods, wherein A is

[chemical structure]

In certain embodiments, the invention relates to any of the aforementioned methods, wherein A is pyrimidin-2-yl, optionally substituted in the four position with a substituent selected from the group consisting of alkyl, halo, haloalkyl, fluoroalkyl, hydroxy, alkoxy, haloalkoxy, fluoroalkyloxy, amino, azido, cyano, and nitro.

In certain embodiments, the invention relates to any of the aforementioned methods, wherein A is

[chemical structure]

In certain embodiments, the invention relates to any of the aforementioned methods, wherein A is biaryl.

In certain embodiments, the invention relates to any of the aforementioned methods, wherein A is 4-(phenyl)phen-1-yl or 4-(2-pyridinyl)phen-1-yl, optionally substituted with 1, 2, 3, 4 or 5 substituents independently selected from the group consisting of alkyl, halo, haloalkyl, fluoroalkyl, hydroxy, alkoxy, haloalkoxy, fluoroalkyloxy, amino, azido, cyano, and nitro.

In certain embodiments, the invention relates to any of the aforementioned methods, wherein A is

[chemical structure]

In certain embodiments, the invention relates to any of the aforementioned methods, wherein R¹ is hydrogen.

In certain embodiments, the invention relates to any of the aforementioned methods, wherein R¹ is alkyl.

In certain embodiments, the invention relates to any of the aforementioned methods, wherein R¹ is haloalkyl.

In certain embodiments, the invention relates to any of the aforementioned methods, wherein R¹ is fluoroalkyl.

In certain embodiments, the invention relates to any of the aforementioned methods, wherein R¹ is methyl.

In certain embodiments, the invention relates to any of the aforementioned methods, wherein R¹ is halomethyl.

In certain embodiments, the invention relates to any of the aforementioned methods, wherein R¹ is fluoromethyl.

In certain embodiments, the invention relates to any of the aforementioned methods, wherein R¹ is ethyl.

In certain embodiments, the invention relates to any of the aforementioned methods, wherein R¹ is haloethyl.

In certain embodiments, the invention relates to any of the aforementioned methods, wherein R¹ is fluoroethyl.

In certain embodiments, the invention relates to any of the aforementioned methods, wherein R² is hydrogen.

In certain embodiments, the invention relates to any of the aforementioned methods, wherein R² is alkyl.

In certain embodiments, the invention relates to any of the aforementioned methods, wherein R² is methyl.

In certain embodiments, the invention relates to any of the aforementioned methods, wherein R² is ethyl.

In certain embodiments, the invention relates to any of the aforementioned methods, wherein R¹ is hydrogen; and R² is hydrogen.

In certain embodiments, the invention relates to any of the aforementioned methods, wherein R¹ is alkyl; and R² is alkyl.

In certain embodiments, the invention relates to any of the aforementioned methods, wherein R¹ is methyl; and R² is methyl.

In certain embodiments, the invention relates to any of the aforementioned methods, wherein R¹ is ethyl; and R² is ethyl.

In certain embodiments, the invention relates to any of the aforementioned methods, wherein Z is ═C(R⁸)—; and R⁸ is hydrogen.

In certain embodiments, the invention relates to any of the aforementioned methods, wherein Z is ═C(R⁸)—; and R⁸ is alkyl.

In certain embodiments, the invention relates to any of the aforementioned methods, wherein Z is ═N—.

In certain embodiments, the invention relates to any of the aforementioned methods, wherein X is

[chemical structure]

In certain embodiments, the invention relates to any of the aforementioned methods, wherein X is

[chemical structure]

In certain embodiments, the invention relates to any of the aforementioned methods, wherein X is

[chemical structure]

In certain embodiments, the invention relates to any of the aforementioned methods, wherein X is heteroaryl.

In certain embodiments, the invention relates to any of the aforementioned methods, wherein X is pyrrolo[1,2-a]pyrazin-3-yl.

In certain embodiments, the invention relates to any of the aforementioned methods, wherein R⁹ is alkyl. In certain embodiments, the invention relates to any of the aforementioned compounds, wherein R⁹ is methyl.

In certain embodiments, the invention relates to any of the aforementioned methods, wherein Y is —CH₂NR³R⁴.

In certain embodiments, the invention relates to any of the aforementioned methods, wherein Y is —CH₂NR³R⁴; and R³ is hydrogen.

In certain embodiments, the invention relates to any of the aforementioned methods, wherein Y is —CH₂NR³R⁴; and R³ is alkyl. In certain embodiments, the invention relates to any of the aforementioned compounds, wherein Y is —CH₂NR³R⁴; and R⁴ is hydrogen.

In certain embodiments, the invention relates to any of the aforementioned methods, wherein Y is —CH₂NR³R⁴; and R⁴ is alkyl.

In certain embodiments, the invention relates to any of the aforementioned methods, wherein Y is —CH₂NR³R⁴; and R⁴ is alkoxyalkyl.

In certain embodiments, the invention relates to any of the aforementioned methods, wherein Y is —CH₂NR³R⁴; R³ is hydrogen; and R⁴ is alkyl.

In certain embodiments, the invention relates to any of the aforementioned methods, wherein Y is —CH₂NR³R⁴; R³ is alkyl; and R⁴ is alkyl.

In certain embodiments, the invention relates to any of the aforementioned methods, wherein Y is —CH₂NR³R⁴; R³ is hydrogen; and R⁴ is alkoxyalkyl.

In certain embodiments, the invention relates to any of the aforementioned methods, wherein Y is —CH₂NR³R⁴; R³ is alkyl; and R⁴ is alkoxyalkyl.

In certain embodiments, the invention relates to any of the aforementioned methods, wherein Y is

[chemical structure]

In certain embodiments, the invention relates to any of the aforementioned methods, wherein Y is —CH₂(N-heterocyclyl), which is optionally substituted with one, two, three, four or five substituents independently selected from the group consisting of alkyl, haloalkyl, fluoroalkyl, halo, hydroxyl, alkoxy, haloalkoxy, fluoroalkoxy, amino and nitro.

In certain embodiments, the invention relates to any of the aforementioned methods, wherein Y is —CH₂ (piperidin-1-yl), —CH₂ (piperazin-1-yl), —CH₂ (hexahydropyrimidin-1-yl), —CH₂(morpholin-1-yl) or —CH₂(1,3-oxazinan-3-yl), which is optionally substituted with one, two, three, four or five substituents independently selected from the group consisting of alkyl, haloalkyl, fluoroalkyl, halo, hydroxyl, alkoxy, haloalkoxy, fluoroalkoxy, amino and nitro.

In certain embodiments, the invention relates to any of the aforementioned methods, wherein Y is —CH₂(piperidin-1-yl) or —CH₂(piperazin-1-yl), which is optionally substituted with one, two, three, four or five substituents independently selected from the group consisting of alkyl, haloalkyl, fluoroalkyl, halo, hydroxyl, alkoxy, haloalkoxy, fluoroalkoxy, amino and nitro.

In certain embodiments, the invention relates to any of the aforementioned methods, wherein Y is

[chemical structure]

[chemical structure]

In certain embodiments, the invention relates to any of the aforementioned methods, wherein Y is —CH₂NH(CH₂)ₙNH(alkyl), —CH₂NH(CH₂)ₙN(alkyl)₂, —CH₂NH(CH₂)ₙN(alkylene), —CH₂N(alkyl)(CH₂)ₙNH(alkyl), —CH₂N(alkyl)(CH₂)ₙN(alkyl)₂ or —CH₂N(alkyl)(CH₂)ₙN(alkylene).

In certain embodiments, the invention relates to any of the aforementioned methods, wherein Y is —CH₂NH(CH₂)ₙO(alkyl) or —CH₂N(alkyl)(CH₂)ₙO(alkyl).

In certain embodiments, the invention relates to any of the aforementioned methods, wherein n is 1.

In certain embodiments, the invention relates to any of the aforementioned methods, wherein n is 2.

In certain embodiments, the invention relates to any of the aforementioned methods, wherein n is 3.

In certain embodiments, the invention relates to any of the aforementioned methods, wherein n is 4.

In certain embodiments, the invention relates to any of the aforementioned methods, wherein Y is

[chemical structure]

In certain embodiments, the invention relates to any of the aforementioned methods, wherein Y is —NR³R⁴.

In certain embodiments, the invention relates to any of the aforementioned methods, wherein Y is —NR³R⁴; and R³ is hydrogen.

In certain embodiments, the invention relates to any of the aforementioned methods, wherein Y is —NR³R⁴; and R³ is alkyl.

In certain embodiments, the invention relates to any of the aforementioned methods, wherein Y is —NR³R⁴; and R⁴ is hydrogen.

In certain embodiments, the invention relates to any of the aforementioned methods, wherein Y is —NR³R⁴; and R⁴ is alkyl.

In certain embodiments, the invention relates to any of the aforementioned methods, wherein Y is —NR³R⁴; R³ is hydrogen; and R⁴ is alkyl.

In certain embodiments, the invention relates to any of the aforementioned methods, wherein Y is —NR³R⁴; R³ is hydrogen; and R⁴ is hydrogen. In certain embodiments, the invention relates to any of the aforementioned compounds, wherein Y is —NR³R⁴; R³ is alkyl; and R⁴ is alkyl.

In certain embodiments, the invention relates to any of the aforementioned methods, wherein Y is —NR⁵NR⁶R⁷ or —NR⁵(N-heterocyclyl).

In certain embodiments, the invention relates to any of the aforementioned methods, wherein Y is —NR⁵NR⁶R⁷; and R⁵ is hydrogen.

In certain embodiments, the invention relates to any of the aforementioned methods, wherein Y is —NR⁵NR⁶R⁷; and R⁵ is alkyl.

In certain embodiments, the invention relates to any of the aforementioned methods, wherein Y is —NR⁵NR⁶R⁷; and R⁵, R⁶ and R⁷ are, independently, hydrogen or alkyl.

In certain embodiments, the invention relates to any of the aforementioned methods, wherein Y is —NR⁵(N-heterocyclyl); and R⁵ is hydrogen.

In certain embodiments, the invention relates to any of the aforementioned methods, wherein Y is —NR⁵(N-heterocyclyl); and R⁵ is alkyl.

In certain embodiments, the invention relates to any of the aforementioned methods, wherein Y is

[chemical structure]

In certain embodiments, the invention relates to any of the aforementioned methods, wherein Z is ═C(R²)—; and the two R² taken together are

[chemical structure]

In certain embodiments, the invention relates to any of the aforementioned methods, wherein Z is ═C(R²)—; and the two R² taken together are

[chemical structure]

In certain embodiments, the invention relates to any of the aforementioned methods, wherein Z is ═C(R²)—; and the two R² taken together are

[chemical structure]

In certain embodiments, the invention relates to any of the aforementioned methods, wherein Z is ═C(R²)—; and the two R² taken together are

[chemical structure]

In certain embodiments, the invention relates to any of the aforementioned methods, wherein Z is ═C(R²)—; and the two R² taken together are

[chemical structure]

In certain embodiments, the invention relates to any of the aforementioned methods, wherein R¹⁰ is hydrogen, alkyl, haloalkyl, fluoroalkyl, alkoxy, alkoxyalkyl, halo or trifluoromethyl.

In certain embodiments, the invention relates to any of the aforementioned methods, wherein R¹⁰ is hydrogen, amino, amido, N-heterocyclyl, aminoalkyl, amidoalkyl, or N-hetrocyclylalkyl.

In certain embodiments, the invention relates to any of the aforementioned methods, wherein R¹⁰ is hydrogen, halo or N-heterocyclyl.

In certain embodiments, the invention relates to any of the aforementioned methods, wherein R¹⁰ is hydrogen, chloro or piperidin-1-yl.

In certain embodiments, the invention relates to any of the aforementioned methods, wherein R¹⁰ is hydrogen or N-heterocyclylalkyl.

In certain embodiments, the invention relates to any of the aforementioned methods, wherein R¹⁰ is hydrogen or piperidin-1-ylmethyl.

In certain embodiments, the invention relates to any of the aforementioned methods, wherein R¹⁰ is hydrogen or alkyl.

In certain embodiments, the invention relates to any of the aforementioned methods, wherein R¹⁰ is hydrogen.

In certain embodiments, the invention relates to any of the aforementioned methods, wherein R¹¹ is hydrogen or alkyl.

In certain embodiments, the invention relates to any of the aforementioned methods, wherein R¹¹ is hydrogen.

In certain embodiments, the invention relates to any of the aforementioned methods, wherein R¹¹ is methyl.

In certain embodiments, the invention relates to any of the aforementioned methods, wherein R¹² is hydrogen.

In certain embodiments, the invention relates to any of the aforementioned methods, wherein R¹² is methyl. In certain embodiments, the invention relates to any of the aforementioned methods, wherein R¹³ is hydrogen.

In certain embodiments, the invention relates to any of the aforementioned methods, wherein exactly one R¹³, and the carbon to which it is bound, is —N═.

In certain embodiments, the invention relates to any of the aforementioned methods, wherein R¹⁴ is hydrogen.

In certain embodiments, the invention relates to any of the aforementioned methods, wherein R¹⁴ is X. In certain embodiments, the invention relates to any of the aforementioned methods, wherein Z is ═C(R²)—; the two R² taken together are

[chemical structure]

and R¹⁰ is hydrogen, halo or N-heterocyclyl.

In certain embodiments, the invention relates to any of the aforementioned methods, wherein Z is ═C(R²)—; the two R² taken together are

[chemical structure]

and R¹⁰ is hydrogen or N-heterocyclylalkyl.

In certain embodiments, the invention relates to any of the aforementioned methods, wherein the first compound is selected from the group consisting of

[chemical structure]

[chemical structure]

[chemical structure]

[chemical structure]

[chemical structure]

[chemical structure]

In certain embodiments, the invention relates to any of the aforementioned methods, wherein the first compound is selected from the group consisting of

[chemical structure]

In certain embodiments, the invention relates to any of the aforementioned methods, wherein the first compound is

[chemical structure]

In certain embodiments, the invention relates to any of the aforementioned methods, wherein the first compound is selected from the group consisting of

[chemical structure]

[chemical structure]

[chemical structure]

[chemical structure]

wherein W is methyl, fluoro, chloro, nitro, methoxy, ethoxy, —SO₂NH₂ or —C(═O)NH₂.

In certain embodiments, the invention relates to any of the aforementioned methods, wherein the first compound is selected from the group consisting of

[chemical structure]

wherein W is alkyl, fluoro, chloro, nitro, methoxy, ethoxy, —SO₂NH₂ or —C(═O)NH₂.

In certain embodiments, the invention relates to any of the aforementioned methods, wherein W is methyl.

In certain embodiments, the invention relates to any of the aforementioned methods, wherein W is fluoro.

In certain embodiments, the invention relates to any of the aforementioned methods, wherein W is chloro.

In certain embodiments, the invention relates to any of the aforementioned methods, wherein W is nitro.

In certain embodiments, the invention relates to any of the aforementioned methods, wherein W is methoxy.

In certain embodiments, the invention relates to any of the aforementioned methods, wherein W is ethoxy.

In certain embodiments, the invention relates to any of the aforementioned methods, wherein W is —SO₂NH₂.

In certain embodiments, the invention relates to any of the aforementioned methods, wherein W is —C(═O)NH₂.

In certain embodiments, the invention relates to any of the aforementioned methods, wherein W is chloro.

In certain embodiments, the invention relates to any of the aforementioned methods, wherein the first compound is selected from the group consisting of

[chemical structure]

[chemical structure]

In certain embodiments, the invention relates to any of the aforementioned methods, wherein the first compound is selected from the group consisting of

[chemical structure]

In certain embodiments, the invention relates to any of the aforementioned methods, wherein the first compound is selected from the group consisting of

[chemical structure]

In certain embodiments, the invention relates to any of the aforementioned methods, wherein the first compound is selected from the group consisting of

[chemical structure]

Many of the compounds used in the methods of the invention may be provided as salts with pharmaceutically compatible counterions (i.e., pharmaceutically acceptable salts). A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like salts. Pharmaceutically acceptable acid addition salts include those formed with mineral acids such as hydrochloric acid and hydrobromic acid, and those formed with organic acids such as maleic acid.

Suitable bases for forming pharmaceutically acceptable salts with acidic functional groups include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl-N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

Certain compounds used in methods of the invention and their salts may exist in more than one crystal form and the invention includes each crystal form and mixtures thereof.

Certain compounds used in methods of the invention and their salts may also exist in the form of solvates, for example hydrates, and the invention includes each solvate and mixtures thereof.

Certain compounds used in methods of the invention may contain one or more chiral centers, and exist in different optically active forms. When compounds of the invention contain one chiral center, the compounds exist in two enantiomeric forms and the invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step may be used to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound used in the methods of the invention contains more than one chiral center, it may exist in diastereoisomeric forms. The diastereoisomeric compounds may be separated by methods known to those skilled in the art, for example chromatography or crystallization and the individual enantiomers may be separated as described above. The invention includes each diastereoisomer of compounds of the invention and mixtures thereof.

Certain compounds used in methods of the invention may exist in different tautomeric forms or as different geometric isomers, and the invention includes each tautomer and/or geometric isomer of compounds of the invention and mixtures thereof.

Certain compounds used in methods of the invention may exist in different stable conformational forms which may be separable. Torsional asymmetry due to restricted rotation about an asymmetric single bond, for example because of steric hindrance or ring strain, may permit separation of different conformers. The invention includes each conformational isomer of compounds of the invention and mixtures thereof.

Certain compounds used in methods of the invention may exist in zwitterionic form and the invention includes each zwitterionic form of compounds of the invention and mixtures thereof.

The invention also includes methods of using pro-drugs. As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a pro-drug would be a compound of the invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial. Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents (e.g., —C(O)₂H or a moiety that contains a carboxylic acid) wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino (C₂-C₃)alkyl (such as (3-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol or amine of a compound of the invention wherein the free hydrogen of a hydroxyl or amine substituent is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyl-oxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, —P(O)(OH)₂, —P(O)(O(C₁-C₆)alkyl)₂ or glycosyl (the radical resulting from detachment of the hydroxyl of the hemiacetal of a carbohydrate).

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

The term “chemically protected form,” as used herein, pertains to a compound in which one or more reactive functional groups are protected from undesirable chemical reactions, that is, are in the form of a protected or protecting group (also known as a masked or masking group). It may be convenient or desirable to prepare, purify, and/or handle the active compound in a chemically protected form.

By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1991), and Protective Groups in Organic Synthesis (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃,—OAc).

For example, an aldehyde or ketone group may be protected as an acetal or ketal, respectively, in which the carbonyl group (C(═O)) is converted to a diether (C(OR)₂), by reaction with, for example, a primary alcohol. The aldehyde or ketone group is readily regenerated by hydrolysis using a large excess of water in the presence of acid.

For example, an amine group may be protected, for example, as an amide (—NRC(═O)R) or a urethane (—NRC(═O)OR), for example, as: a methyl amide (—NHC(═O)CH₃); a benzyloxy amide (—NHC(═O)OCH₂C₆H₅NHCbz); as a t-butoxy amide (—NHC(═O)OC(CH₃)₃,—NHBoc); a 2-biphenyl-2-propoxy amide (—NHC(═O)OC(CH₃)₂C₆H₄C₆H₅NHBoc), as a 9-fluorenylmethoxy amide (—NHFmoc), as a 6-nitroveratryloxy amide (—NHNvoc), as a 2-trimethylsilylethyloxy amide (—NHTeoc), as a 2,2,2-trichloroethyloxy amide (—NHTroc), as an allyloxy amide (—NHAlloc), as a 2-(phenylsulfonyl)ethyloxy amide (—NHPsec); or, in suitable cases (e.g., cyclic amines), as a nitroxide radical.

For example, a carboxylic acid group may be protected as an ester or an amide, for example, as: a benzyl ester; a t-butyl ester; a methyl ester; or a methyl amide.

For example, a thiol group may be protected as a thioether (—SR), for example, as: a benzyl thioether; or an acetamidomethyl ether (—SCH₂NHC(═O)CH₃).

### Combination Therapy

In certain embodiments, the first compound and the second compound may be administered simultaneously or sequentially. When the compounds are administered simultaneously they can be administered in the same or separate formulations, but are administered at the same time. The compounds are administered sequentially with one another when the administration of the first compound and the administration of the second compound are temporally separated. The separation in time between the administrations of these compounds may be a matter of minutes or it may be longer.

One aspect of the present invention relates to combination therapy. This type of therapy is advantageous because the co-administration of active ingredients achieves a therapeutic effect that is greater than the therapeutic effect achieved by administration of only a single therapeutic agent.

In certain embodiments, the co-administration of two or more therapeutic agents achieves a therapeutic effect that is greater than the therapeutic effect achieved by administration of only a single therapeutic agent. In this regard, the combination therapies are efficacious. The therapeutic effect of one therapeutic agent is augmented by the co-administration of another therapeutic agent.

In certain embodiments, the co-administration of two or more therapeutic agents achieves a therapeutic effect that is equal to about the sum of the therapeutic effects achieved by administration of each single therapeutic agent. In these embodiments, the combination therapies are said to be “additive.”

In certain embodiments, the co-administration of two or more therapeutic agents achieves a synergistic effect, i.e., a therapeutic effect that is greater than the sum of the therapeutic effects of the individual components of the combination.

The active ingredients that comprise a combination therapy may be administered together via a single dosage form or by separate administration of each active agent. In certain embodiments, the first and second therapeutic agents are administered in a single dosage form. In certain embodiments, the first and second therapeutic agents are administered in a single dosage form. The agents may be formulated into a single tablet, pill, capsule, or solution for parenteral administration and the like.

In certain embodiments, the therapeutic agents are administered in a single dosage form, wherein each individual therapeutic agent is isolated from the other therapeutic agent(s). Formulating the dosage forms in such a way assists in maintaining the structural integrity of potentially reactive therapeutic agents until they are administered. A formulation of this type may be useful during production and for long-term storage of the dosage form. In certain embodiments, the therapeutic agents may comprise segregated regions or distinct caplets or the like housed within a capsule. In certain embodiments, the therapeutic agents are provided in isolated layers comprised by a tablet.

Alternatively, the therapeutic agents may be administered as separate compositions, e.g., as separate tablets or solutions. One or more active agent may be administered at the same time as the other active agent(s) or the active agents may be administered intermittently. The length of time between administrations of the therapeutic agents may be adjusted to achieve the desired therapeutic effect. In certain instances, one or more therapeutic agent(s) may be administered only a few minutes (e.g., about 1, 2, 5, 10, 30, or 60 min) after administration of the other therapeutic agent(s). Alternatively, one or more therapeutic agent(s) may be administered several hours (e.g., about 2, 4, 6, 10, 12, 24, or 36 hr) after administration of the other therapeutic agent(s). In certain embodiments, it may be advantageous to administer more than one dosage of one or more therapeutic agent(s) between administrations of the remaining therapeutic agent(s). For example, one therapeutic agent may be administered at 2 hours and then again at 10 hours following administration of the other therapeutic agent(s). Importantly, it is required that the therapeutic effects of each active ingredient overlap for at least a portion of the duration of each therapeutic agent so that the overall therapeutic effect of the combination therapy is attributable in part to the combined or synergistic effects of the combination therapy.

The dosage of the active agents will generally be dependent upon a number of factors including pharmacodynamic characteristics of each agent of the combination, mode and route of administration of active agent(s), the health of the patient being treated, the extent of treatment desired, the nature and kind of concurrent therapy, if any, and the frequency of treatment and the nature of the effect desired. In general, dosage ranges of the active agents often range from about 0.001 to about 250 mg/kg body weight per day. For a normal adult having a body weight of about 70 kg, a dosage in the range of from about 0.1 to about 25 mg/kg body weight is typically preferred. However, some variability in this general dosage range may be required depending upon the age and weight of the subject being treated, the intended route of administration, the particular agent being administered and the like. Since two or more different active agents are being used together in a combination therapy, the potency of each agent and the interactive effects achieved using them together must be considered. Importantly, the determination of dosage ranges and optimal dosages for a particular mammal is also well within the ability of one of ordinary skill in the art having the benefit of the instant disclosure.

In certain embodiments, it may be advantageous for the pharmaceutical combination to have a relatively large amount of the first component compared to the second component. In certain instances, the ratio of the first active agent to second active agent is about 100:1, 90:1, 80:1, 70:1, 60:1, 50:1, 40:1, 30:1, 20:1, 15:1, 10:1, 9:1, 8:1, 7:1, 6:1, or 5:1. In certain embodiments, it may be preferable to have a more equal distribution of pharmaceutical agents. In certain instances, the ratio of the first active agent to the second active agent is about 4:1, 3:1, 2:1, 1:1, 1:2, 1:3, or 1:4. In certain embodiments, it may be advantageous for the pharmaceutical combination to have a relatively large amount of the second component compared to the first component. In certain instances, the ratio of the second active agent to the first active agent is about 30:1, 20:1, 15:1, 10:1, 9:1, 8:1, 7:1, 6:1, or 5:1. In certain instances, the ratio of the second active agent to first active agent is about 100:1, 90:1, 80:1, 70:1, 60:1, 50:1, or 40:1. Importantly, a composition comprising any of the above-identified combinations of first therapeutic agent and second therapeutic agent may be administered in divided doses about 1, 2, 3, 4, 5, 6, or more times per day or in a form that will provide a rate of release effective to attain the desired results. In one embodiment, the dosage form contains both the first and second active agents. In one embodiment, the dosage form only has to be administered one time per day and the dosage form contains both the first and second active agents.

For example, a formulation intended for oral administration to humans may contain from about 0.1 mg to about 5 g of the first therapeutic agent and about 0.1 mg to about 5 g of the second therapeutic agent, both of which are compounded with an appropriate and convenient amount of carrier material varying from about 5 to about 95 percent of the total composition. Unit dosages will generally contain between about 0.5 mg to about 1500 mg of the first therapeutic agent and 0.5 mg to about 1500 mg of the second therapeutic agent. In a preferred embodiment, the dosage is about 25 mg, 50 mg, 100 mg, 200 mg, 300 mg, 400 mg, 500 mg, 600 mg, 800 mg, or 1000 mg, etc., up to about 1500 mg of the first therapeutic agent. In a preferred embodiment, the dosage is about 25 mg, 50 mg, 100 mg, 200 mg, 300 mg, 400 mg, 500 mg, 600 mg, 800 mg, or 1000 mg, etc., up to about 1500 mg of the second therapeutic agent.

Dosage amount and interval may be adjusted on an individual or group basis to provide plasma levels of a particular active moiety or moieties sufficient to maintain the modulating effects or minimal effective concentration (MEC) of each of them. The MEC will vary for each compound and individual, but it can be estimated from in vitro data. Dosages necessary to achieve the MEC will depend on individual characteristics and route of administration. However, HPLC assays or bioassays can be used to determine plasma concentrations. In certain embodiments, the dose may be increased. Moreover, a long-term treatment regimen may include alternating period of increasing and decreasing dosage with respect to a particular compound or compounds.

The term “synergistic” refers to a combination which is more effective than the additive effects of any two or more single agents. A synergistic effect permits the effective treatment of a disease using lower amounts (doses) of individual therapy. The lower doses result in lower toxicity without reduced efficacy. In addition, a synergistic effect can result in improved efficacy. Finally, synergy may result in an improved avoidance or reduction of disease as compared to any single therapy.

Combination therapy can allow for the product of lower doses of the first therapeutic or the second therapeutic agent (referred to as “apparent one-way synergy” herein), or lower doses of both therapeutic agents (referred to as “two-way synergy” herein) than would normally be required when either drug is used alone.

Combination therapy can allow for the product of lower doses of any one of the therapeutic agents (referred to as “apparent one-way synergy” herein), or lower doses of all therapeutic agents than would normally be required when any drug is used alone.

In certain embodiments, the synergism exhibited between one or more therapeutic agent(s) and the remaining therapeutic agent(s) is such that the dosage of one of the therapeutic agents would be sub-therapeutic if administered without the dosage of the other therapeutic agents.

The terms “augmentation” or “augment” refer to combinations where one of the compounds increases or enhances therapeutic effects of another compound or compounds administered to a patient. In some instances, augmentation can result in improving the efficacy, tolerability, or safety, or any combination thereof, of a particular therapy.

In certain embodiments, the present invention relates to a pharmaceutical composition comprising a therapeutically effective dose of one or more therapeutic agent(s) together with a dose of another therapeutic agent effective to augment the therapeutic effect of the one or more therapeutic agent(s). In other embodiments, the present invention relates to methods of augmenting the therapeutic effect in a patient of one or more therapeutic agent(s) by administering another therapeutic agent to the patient.

In certain preferred embodiments, the invention is directed in part to synergistic combinations of one or more therapeutic agent(s) in an amount sufficient to render a therapeutic effect together with the remaining therapeutic agent(s). For example, in certain embodiments a therapeutic effect is attained which is at least about 2 (or at least about 4, 6, 8, or 10) times greater than that obtained with the dose of the one or more therapeutic agent(s) alone. In certain embodiments, the synergistic combination provides a therapeutic effect which is up to about 20, 30 or 40 times greater than that obtained with the dose of the one or more therapeutic agent(s) alone. In such embodiments, the synergistic combinations display what is referred to herein as an “apparent one-way synergy”, meaning that the dose of the remaining therapeutic agent(s) synergistically potentiates the effect of the one or more therapeutic agent(s), but the dose of the one or more therapeutic agent(s) does not appear to significantly potentiate the effect of the remaining therapeutic agent(s).

In certain embodiments, the combination of active agents exhibits two-way synergism, meaning that the second therapeutic agent potentiates the effect of the first therapeutic agent, and the first therapeutic agent potentiates the effect of the second therapeutic agent. Thus, other embodiments of the invention relate to combinations of a second therapeutic agent and a first therapeutic agent where the dose of each drug is reduced due to the synergism between the drugs, and the therapeutic effect derived from the combination of drugs in reduced doses is enhanced. The two-way synergism is not always readily apparent in actual dosages due to the potency ratio of the first therapeutic agent to the second therapeutic agent. For instance, two-way synergism can be difficult to detect when one therapeutic agent displays much greater therapeutic potency relative to the other therapeutic agent.

The synergistic effects of combination therapy may be evaluated by biological activity assays. For example, the therapeutic agents are mixed at molar ratios designed to give approximately equipotent therapeutic effects based on the EC₉₀ values. Then, three different molar ratios are used for each combination to allow for variability in the estimates of relative potency. These molar ratios are maintained throughout the dilution series. The corresponding monotherapies are also evaluated in parallel to the combination treatments using the standard primary assay format. A comparison of the therapeutic effect of the combination treatment to the therapeutic effect of the monotherapy gives a measure of the synergistic effect. Analysis of synergism, additivity, or antagonism can be determined by analysis of the aforementioned data using the CalcuSyn™ program (Biosoft, Inc.). This program evaluates drug interactions by use of the widely accepted method of Chou and Talalay combined with a statistically evaluation using the Monte Carlo statistical package. The data are displayed in several different formats including median-effect and dose-effects plots, isobolograms, and combination index [CI] plots with standard deviations. For the latter analysis, a CI greater than 1.0 indicates antagonism and a CI less than 1.0 indicates synergism.

Compositions of the invention present the opportunity for obtaining relief from moderate to severe cases of disease. Due to the synergistic or additive or augmented effects provided by the inventive combination of the first and second therapeutic agent, it may be possible to use reduced dosages of each of therapeutic agent. Due to the synergistic or additive or augmented effects provided by the inventive combination of the first and second therapeutic agents, it may be possible to use reduced dosages of each of therapeutic agent. By using lesser amounts of drugs, the side effects associated with each may be reduced in number and degree. Moreover, the inventive combinations avoid side effects to which some patients are particularly sensitive.

### Pharmaceutical Compositions

The invention provides pharmaceutical compositions for use in treating or preventing tauopathies in vitro or in vivo, or for enhancing protein degradation in vitro or in vivo, wherein the composition comprises inhibitors of Usp14 and inhibitors of Hsp70. In one aspect, the invention provides pharmaceutically acceptable compositions which comprise a therapeutically-effective amount of one or more of the compounds described above, formulated together with one or more pharmaceutically acceptable carriers (additives) and/or diluents. In another aspect, the agents of the invention can be administered as such, or administered in mixtures with pharmaceutically acceptable carriers and can also be administered in conjunction with other agents. Conjunctive therapy thus includes sequential, simultaneous and separate, or co-administration of one or more compound of the invention, wherein the therapeutic effects of the first administered has not entirely disappeared when the subsequent compound is administered.

As described in detail below, the pharmaceutical compositions of the invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

As set out above, in certain embodiments, agents of the invention may be compounds containing a basic functional group, such as amino or alkylamino, and are, thus, capable of forming pharmaceutically-acceptable salts with pharmaceutically-acceptable acids. These salts can be prepared in situ in the administration vehicle or the dosage form manufacturing process, or through a separate reaction of a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like (see, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the invention may be compounds containing one or more acidic functional groups and, thus, are capable of forming pharmaceutically-acceptable salts with pharmaceutically-acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations of the compounds of the invention may be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the agent which produces a therapeutic effect.

In certain embodiments, a formulation of the invention comprises an excipient, including, but not limited to, cyclodextrins, liposomes, micelle forming agents, e.g., bile acids, and polymeric carriers, e.g., polyesters and polyanhydrides; and an agent of the invention. In certain embodiments, an aforementioned formulation renders orally bioavailable an agent of the invention.

Methods of preparing these formulations or compositions may include the step of bringing into association a compound of the invention with the carrier and, optionally, one or more accessory ingredients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound of the invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions of the invention may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

Exemplary formulations comprising agents of the invention are determined based on various properties including, but not limited to, chemical stability at body temperature, functional efficiency time of release, toxicity and optimal dose.

The preparations of the invention may be given orally, parenterally, topically, or rectally. They are of course given in forms suitable for each administration route. For example, they are administered in tablets or capsule form, by injection, inhalation, eye lotion, ointment, suppository, administration by injection, infusion or inhalation; topical by lotion or ointment; and rectal by suppositories.

Regardless of the route of administration selected, the compounds of the invention, which may be used in a suitable hydrated form, and/or the pharmaceutical compositions of the invention, are formulated into pharmaceutically-acceptable dosage forms by conventional methods known to those of skill in the art.

### EXEMPLIFICATION

The invention now being generally described, it will be more readily understood by reference to the following examples, which are included merely for purposes of illustration of certain aspects and embodiments of the invention, and are not intended to limit the invention.

### Example 1

### Synergistic Effect of Usp 14 Inhibitor and Hsp70 Inhibitor

The synergistic effect of IU1-47 and JG48 degradation of tau over different time frames, in different doses, via different methods of tau visualization, and in different forms of tau (mutant/wild-type, rodent/human) are shown in FIGS. 5-8. The synergistic effect of IU1-47 and MKT-077 is shown in FIG. 1.

In addition, cell viability was measured with CellTiter-Glo, which measures ATP levels. Cells remained viable over a variety of concentrations of IU1-47 and JG48. See FIG. 9.

### EQUIVALENTS

While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entirety as if each individual publication or patent was specifically and individually indicated to be incorporated by reference. In case of conflict, the present application, including any definitions herein, will control.

We claim:

1. A method of treating or preventing a tauopathy or a trinucleotide repeat disorder in a subject comprising co-administering to the subject
an effective amount of a first compound, or a pharmaceutically acceptable salt, solvate, hydrate, prodrug, chemically-protected form, enantiomer or stereoisomer thereof, and
an effective amount of a second compound, or a pharmaceutically acceptable salt, solvate, hydrate, prodrug, chemically-protected form, enantiomer or stereoisomer thereof,
wherein the first compound inhibits Usp14 and the second compound inhibits Hsp70.

2. The method of claim 1, wherein the method is a method of treating or preventing a tauopathy; and the tauopathy is selected from the group consisting of Alzheimer's Disease (AD), progressive supranuclear palsy, post-encephalitic parkinsonism (PEP), parkinsonism-dementia complex of Guam (PDC Guam), Guadeloupean Parkinsonism, Down's Syndrome, Familial British Dementia, Familial Danish Dementia, Myotonic Dystrophy, Niemann-Pick type C, dementia pugilistica (chronic traumatic encephalopathy), frontotemporal dementia, Parkinson's Disease, Lytico-Bodig disease, tangle-predominant dementia, ganglioglioma, gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), and frontotemporal lobar degeneration.

3. The method of claim 1, wherein the method is a method of treating or preventing a trinucleotide repeat disorder; and the trinucleotide repeat disorder is a polyglutamine disease selected from the group consisting of dentatorubropallidoluysian atrophy, Huntington's disease, spinobulbar muscular atrophy, and spinocerebellar ataxia.

4. The method of claim 1, wherein the first compound is represented by Formula I [chemical structure]
wherein, independently for each occurrence,
A is aryl, heteroaryl, carbocyclyl, heterocyclyl, or biaryl;
R¹ is hydrogen, alkyl, haloalkyl, fluoroalkyl, lower alkoxy, halo or trifluoromethyl;
G is —N═ or —C(R²)═;
Z is ═C(R⁸)—, ═C(R²)— or ═N—;
R² is hydrogen, alkyl, haloalkyl, fluoroalkyl, lower alkoxy, halo or trifluoromethyl; or, when G is —C(R²)═ and Z is ═C(R²)—, the two R² taken together are [chemical structure]
X is [chemical structure]
or heteroaryl;
Y is —CH₂NR³R⁴, —CH₂(N-heterocyclyl), —CH₂NH(CH₂)ₙNH(alkyl), —CH₂NH(CH₂)ₙN(alkyl)₂, —CH₂NH(CH₂)ₙ(N-heterocyclyl), —CH₂N(alkyl)(CH₂)ₙNH(alkyl), —CH₂N(alkyl)(CH₂)ₙN(alkyl)₂, —CH₂N(alkyl)(CH₂)ₙ(N-heterocyclyl), —CH₂NH(CH₂)ₙO(alkyl), —CH₂N(alkyl)(CH₂)ₙO(alkyl), —NR³R⁴, —NR⁵NR⁶R⁷, —NR⁵(N-heterocyclyl), or —N-heterocyclyl;
n is 1, 2, 3 or 4;
R³ is hydrogen, alkyl, substituted alkyl, alkoxyalkyl, haloalkyl, fluoroalkyl, aryl, aralkyl, heteroaryl, or heteroaralkyl;
R⁴ is hydrogen, alkyl, substituted alkyl, alkoxyalkyl, haloalkyl, fluoroalkyl, aryl, aralkyl, heteroaryl, or heteroaralkyl;
R⁵ is hydrogen, alkyl, substituted alkyl, alkoxyalkyl, haloalkyl, fluoroalkyl, aryl, aralkyl, heteroaryl, or heteroaralkyl;
R⁶ is hydrogen, alkyl, substituted alkyl, alkoxyalkyl, haloalkyl, fluoroalkyl, aryl, aralkyl, heteroaryl, or heteroaralkyl;
R⁷ is hydrogen, alkyl, substituted alkyl, alkoxyalkyl, haloalkyl, fluoroalkyl, aryl, aralkyl, heteroaryl, or heteroaralkyl;
R⁸ is hydrogen, alkyl, substituted alkyl, alkoxyalkyl, haloalkyl, fluoroalkyl, aryl, aralkyl, heteroaryl, or heteroaralkyl;
R⁹ is alkyl; or two R⁹ taken together with the nitrogen to which they are bound are an N-heterocyclyl group; and
R¹⁰ is hydrogen, alkyl, haloalkyl, fluoroalkyl, alkoxy, alkoxyalkyl, halo, trifluoromethyl, sulfoxymethyl, sulfonamido, amino, amido, N-heterocyclyl, aminoalkyl, amidoalkyl, or N-hetrocyclylalkyl.

5. The method of claim 1, wherein the first compound is

[chemical structure]

6. The method of claim 1, wherein the second compound is represented by Formula III [chemical structure]
wherein
A′ is a substituted heteroaromatic moiety;
B′ is a substituted heteroaromatic moiety; and
C′ is a substituted heteroaromatic moiety.

7. The method of claim 1, wherein the second compound is selected from the group consisting of MKT-077, JG48, 2-phenylethyenesulfonamide, 5′-O-[(4-cyanophenyl)methyl]-8-[[(3,4-dichlorophenyl)methyl]amino]-adenosine, methylene blue, azure C, and myricetin.

8. The method of claim 1, wherein the second compound is selected from the group consisting of

[chemical structure]

[chemical structure]

[chemical structure]

9. The method of claim 1, wherein the second compound is

[chemical structure]

10. A method of enhancing degradation of a protein in a cell, comprising contacting the cell with
an effective amount of a first compound, or a pharmaceutically acceptable salt, solvate, hydrate, prodrug, chemically-protected form, enantiomer or stereoisomer thereof, and
an effective amount of a second compound, or a pharmaceutically acceptable salt, solvate, hydrate, prodrug, chemically-protected form, enantiomer or stereoisomer thereof,
wherein the first compound inhibits Usp14 and the second compound inhibits Hsp70.

11. The method of claim 10, wherein the protein is selected from the group consisting of Tau and polyglutamine.

12. The method of claim 10, wherein the first compound is represented by Formula I [chemical structure]
wherein, independently for each occurrence,
A is aryl, heteroaryl, carbocyclyl, heterocyclyl, or biaryl;
R¹ is hydrogen, alkyl, haloalkyl, fluoroalkyl, lower alkoxy, halo or trifluoromethyl;
G is —N═ or —C(R²)═;
Z is ═C(R⁸)—, ═C(R²)— or ═N—;
R² is hydrogen, alkyl, haloalkyl, fluoroalkyl, lower alkoxy, halo or trifluoromethyl; or, when G is —C(R²)═ and Z is ═C(R²)—, the two R² taken together are [chemical structure]
X is [chemical structure]
or heteroaryl;
Y is —CH₂NR³R⁴, —CH₂(N-heterocyclyl), —CH₂NH(CH₂)ₙNH(alkyl), —CH₂NH(CH₂)ₙN(alkyl)₂, —CH₂NH(CH₂)ₙ(N-heterocyclyl), —CH₂N(alkyl)(CH₂)ₙNH(alkyl), —CH₂N(alkyl)(CH₂)ₙN(alkyl)₂, —CH₂N(alkyl)(CH₂)ₙ(N-heterocyclyl), —CH₂NH(CH₂)ₙO(alkyl), —CH₂N(alkyl)(CH₂)ₙO(alkyl), —NR³R⁴, —NR⁵NR⁶R⁷, —NR⁵(N-heterocyclyl), or —N-heterocyclyl;
n is 1, 2, 3 or 4;
R³ is hydrogen, alkyl, substituted alkyl, alkoxyalkyl, haloalkyl, fluoroalkyl, aryl, aralkyl, heteroaryl, or heteroaralkyl;
R⁴ is hydrogen, alkyl, substituted alkyl, alkoxyalkyl, haloalkyl, fluoroalkyl, aryl, aralkyl, heteroaryl, or heteroaralkyl;
R⁵ is hydrogen, alkyl, substituted alkyl, alkoxyalkyl, haloalkyl, fluoroalkyl, aryl, aralkyl, heteroaryl, or heteroaralkyl;
R⁶ is hydrogen, alkyl, substituted alkyl, alkoxyalkyl, haloalkyl, fluoroalkyl, aryl, aralkyl, heteroaryl, or heteroaralkyl;
R⁷ is hydrogen, alkyl, substituted alkyl, alkoxyalkyl, haloalkyl, fluoroalkyl, aryl, aralkyl, heteroaryl, or heteroaralkyl;
R⁸ is hydrogen, alkyl, substituted alkyl, alkoxyalkyl, haloalkyl, fluoroalkyl, aryl, aralkyl, heteroaryl, or heteroaralkyl;
R⁹ is alkyl; or two R⁹ taken together with the nitrogen to which they are bound are an N-heterocyclyl group; and
R¹⁰ is hydrogen, alkyl, haloalkyl, fluoroalkyl, alkoxy, alkoxyalkyl, halo, trifluoromethyl, sulfoxymethyl, sulfonamido, amino, amido, N-heterocyclyl, aminoalkyl, amidoalkyl, or N-hetrocyclylalkyl.

13. The method of claim 10, wherein the first compound is

[chemical structure]

14. The method of claim 10, wherein the second compound is represented by Formula III [chemical structure]
wherein
A′ is a substituted heteroaromatic moiety;
B′ is a substituted heteroaromatic moiety; and
C′ is a substituted heteroaromatic moiety.

15. The method of claim 10, wherein the second compound is selected from the group consisting of MKT-077, JG48, 2-phenylethyenesulfonamide, 5′-O-[(4-cyanophenyl)methyl]-8-[[(3,4-dichlorophenyl)methyl]amino]-adenosine, methylene blue, azure C, and myricetin.

16. The method of claim 10, wherein the second compound is selected from the group consisting of

[chemical structure]

[chemical structure]

[chemical structure]

17. The method of claim 10, wherein the second compound is [chemical structure]
